(19) United States
(12) Patent Application Publication
(10) Pub. No.: US 2003/0190375 A1
(43) Pub. Date: Oct. 9, 2003

(54) Therapeutical use of sophora flavescens or sophora subprostrata extracts

(76) Inventors: Clemens Erdelmeier, Karlsruhe (DE); Egon Koch, Karlsruhe (DE)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/312,442

(22) PCT Filed: Jun. 29, 2001
(86) PCT No.: PCT/DE01/02461

(30) Foreign Application Priority Data
Jun. 29, 2000 (DE) 100 31 650.6

(51) Int. Cl. A61K 35/78

(52) U.S. Cl. 424/725

(57) ABSTRACT

The invention relates to the use of extracts from Sophora flavescens or Sophora subprostrata< for the prophylaxis and therapy of pathological conditions that are caused by estrogen deficiency or by other dysregulations of the sex hormone metabolism, especially of the estrogen metabolism.

The present invention relates to the use of extracts of Sophora flavescens or Sophora subprostrata for the prophylaxis and treatment of pathological conditions caused by oestrogen deficiency or by dysregulations to sex-hormone-related metabolism, in particular oestrogen metabolism.

Irregular menstrual cycles appear in women from around the age of 40, marking the onset of the menopause. This phase of changes in the endocrine system, known as the climacteric, may persist for a decade or more. It results from the exhaustion of follicle growth and reduced responses to gonadotrophin. As a consequence of this follicle deficit, production of oestrogen declines and eventually stops altogether. In association with this, a variety of symptoms may develop, such as hot flushes, depression, anxiety, mental confusion, insomnia etc. In addition to these, serious health problems may arise as a result of the decline in oestrogen production, such as osteoporosis, cardiac insufficiency, cerebral infarction (strokes) and cancer.

Although hormone replacement therapy with oestrogens for the relief of climacteric-related complaints is highly effective, numerous studies show that an increased risk of breast cancer and cancer of the womb, cardiovascular diseases and changes to liver metabolism can be linked to this. The risk of cancer can be reduced by administering progestines. However, the protective effect of progestines appears to fade with long-term use and it is thought that hormone replacement therapy (HRT) is linked with a 35-40% increased risk of breast cancer. Given the side effects, also distinct doubts as to the reliability of this type of treatment, the use of HRT medicine in post-menopausal women over age 40 tends to be refused. It is estimated that only around 20% of post-menopausal American women receive HRT, of whom just 40% continue with the treatment for longer than one year.

Substances that have an oestrogen-agonistic effect in humans or animals, or which interact with oestrogen, have been discovered in many plants. At least 20 different groups of substances with oestrogenic/anti-oestrogenic properties, coming from over 300 plants of more than 16 families of plants, have thus far been found. The majority of the known phyto-oestrogens belong to the isoflavone, lignane or cumestane families. Only recently, a further highly potent phyto-oestrogen has been identified—8-prenylnaringenine (S. R. Milligan et al; Journal of Clin. Endocrinol. Metab. 84, 2249-2252 [1999]). The oestrogenic potency of 8-prenylnaringenine in vitro was tested by stimulating alkaline phosphatase in Ishikawa-Var-I cells. In so doing, it was found that 8-penylnaringenine was significantly more active than the long-familiar phyto-oestrogens such as cumesterol, genistone or daidzin, and produced only a slightly weaker effect than 17β oestradiol.

Controlled epidemiological studies on populations in the USA, Europe, China and Japan have shown that a close negative correlation exists between the uptake of phyto-oestrogens with food and the appearance of various tumours, especially breast cancer and prostate cancer. The inhibiting effects of phyto-oestrogens on the growth of mammary and prostate tumour cells have also been confirmed in animal experiments. The preventive properties of phyto-oestrogens are clearly based on a large number of different biological characteristics that discriminate between these combinations of synthetic and natural oestrogens. Depending on the dosage and the endogenous hormone status, phyto-oestrogens also have oestrogenic or even anti-oestrogenic effects. Other biological effects include, for example, the inhibition of tyrosinkinases, DNA topoisomerases, ribosomal S6 kinase, ornithin-decaroxylase, aromatase or 5α reductase. Moreover, phyto-oestrogens frequently have anti-oxidising properties and it is clear that the sum total of all these various actions plays a part in inhibiting the appearance and growth of tumours (Tham et al, Journal of Clin. Endocrin. Metab. 83, 2223-2235 [1998]).

The object underlying the present invention is to suggest the use of plant extracts that are suitable for the prophylaxis and treatment of pathological conditions that are caused by an oestrogen deficiency or by a dysregulation of the sex-related hormone metabolism, in particular oestrogen metabolism.

This object is solved according to the invention by the use of extracts of Sophora flavescens or Sophora subprostrata according to claims 1-3 and by the methods according to claims 4-7.

Several Sophora species (Fabacaea) are known from traditional Chinese medicine whose roots can be used medicinally. These are Sophora flavescens, S. subprostrata, S. alopeculoides, S. japonica, S. tonkinensis, S. tomentosa, S. moorcroftiana and S. leachiana. Of these, S. flavescens turns out to be of special significance. For this reason, it is found in the Chinese Pharmacopœia (Sophorae flavescentis radix, “Kushen”, Pharmacopoeia of Chinese Medicine 9/1997) and is traditionally used for the treatment of diarrhoea, gastro-intestinal haemorrhaging and eczema (W. Tang, G. Eisenbrand, Chinese Drugs of Plant Origin, Springer Verlag, Berlin 1992).

S. flavescens roots, as well as those of other Sophora species, contain a sequence of chinolizidine alkaloids as one of their main ingredients. The Chinese Pharmacopoeia stipulates that the total titrimetric content of alkaloids may not fall below 2%. The main alkaloids are matrine and oxymatrine.

Besides the alkaloids, a large number of flavones and related compounds and triterpensaponines were found in the roots of S. flavescens, as well as in quite a few other Sophora species.

A variety of pharmacological properties have been described for Sophora flavescens, also for individual ingredients or groups of ingredients. Alkaloids, in particular, have been thought to account for anti-arrhythmic, anti-histamine and anti-tussive effects, also for anti-ulcer effects (Tang and Eisenbrand, see above). Anti-neoplastic effects and immune-suppressant properties have also been observed.

M. Kuroyanagi et al, Journal of Natural Products 1999, 62 (12) 1595-1599, report on anti-bacterial and anti-androgenic flavonoids from Sophora flavescens. It was found that virtually all prenylflavone derivatives isolated from Sophora flavescens showed an anti-androgenic effect.

W. M. Mazur et al, Nutritional Biochemistry 1998, 9, 193-200, report on the quantitative determination of the isoflavones formononetin, biochanine A, daidzein, genistone and cumestrol and the lignanes secoisolariciresinol and matairesinol in leguminosae seeds, when seeds of Sophora japonica were investigated among others.

In toxicology, S. flavescens and S. japonica are classified as toxic. In particular, alkaloids of the matrine type are thought to be poisonous (T. Blaszczyk, Deutsche Apotheke Zeitung 2001, 141 [14], 1687-1696). In clinical samples, toxic effects such as severe palpitations, dyspnoea and spasms were observed in the case of Sophora alkaloids. In animal studies, toxic effects could be shown for oxymatrine (K.-C. Huang, The Pharmacology of Chinese Herbs, CRC press, Boca Raton, 1993).

Species of Sophora particularly favoured for the present invention are Sophora flavescens and Sophora subprostrata, for their aqueous-alcoholic root extracts were found to have surprisingly powerful oestrogenic effects in the context of this invention. It was possible to enrich these aqueous-alcoholic extracts by further separation and purification. It turned out that pharmacological effects observed were caused by the interaction of the isoflavones present in the extract: genistone and daidzein, flavones such as 8-prenylnaringenine, kushenol X, 8-prenylcamphorol, leachianon G and kushenol E, chalcones and pterocarpanes such as macchian and macchian glucoside, as well as other unidentified flavonide-like compounds. In this connection, the chalcone newly discovered by the inventors: 2,4,4′,6′ tetrahydroxy-3′-lavandulyl-2′-methoxychalcone of the formula below should also be mentioned.

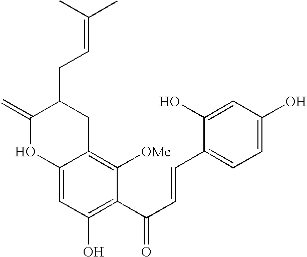

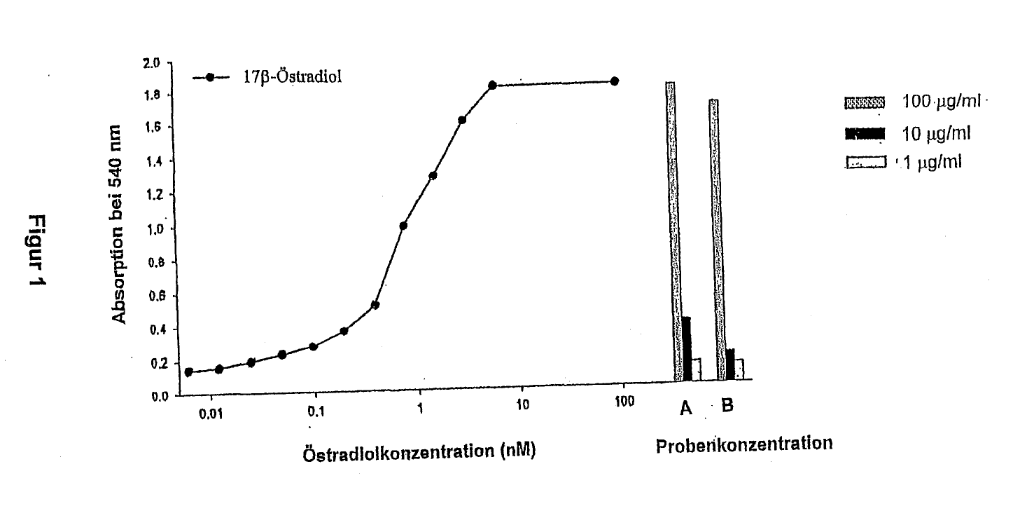

FIG. 1 illustrates the oestrogenic action of 70% [v/v] (62% [w/w]) ethanol extracts (according to examples 1a) and 2a)) from Sophora flavescens and Sophora subprostrata in a yeast assay, compared with 17β oestradiol.

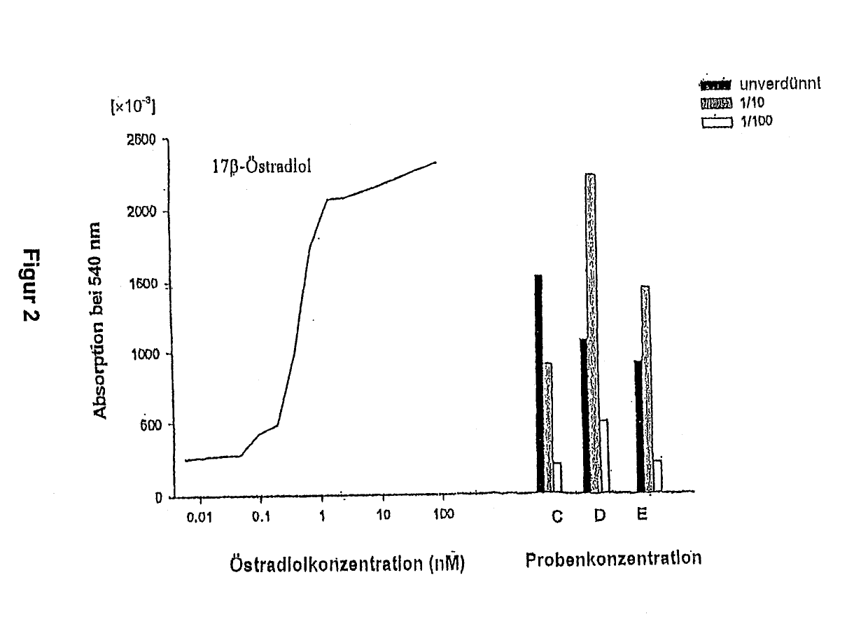

FIG. 2 illustrates the oestrogenic action of extracts according to the invention (70% [v/v] or 62% [w/w] ethanol extracts after distribution with ethylacetate; according to examples 1b) and 2b)) from Sophora flavescens and Sophora subprostrata in a yeast assay, compared with 17β oestradiol.

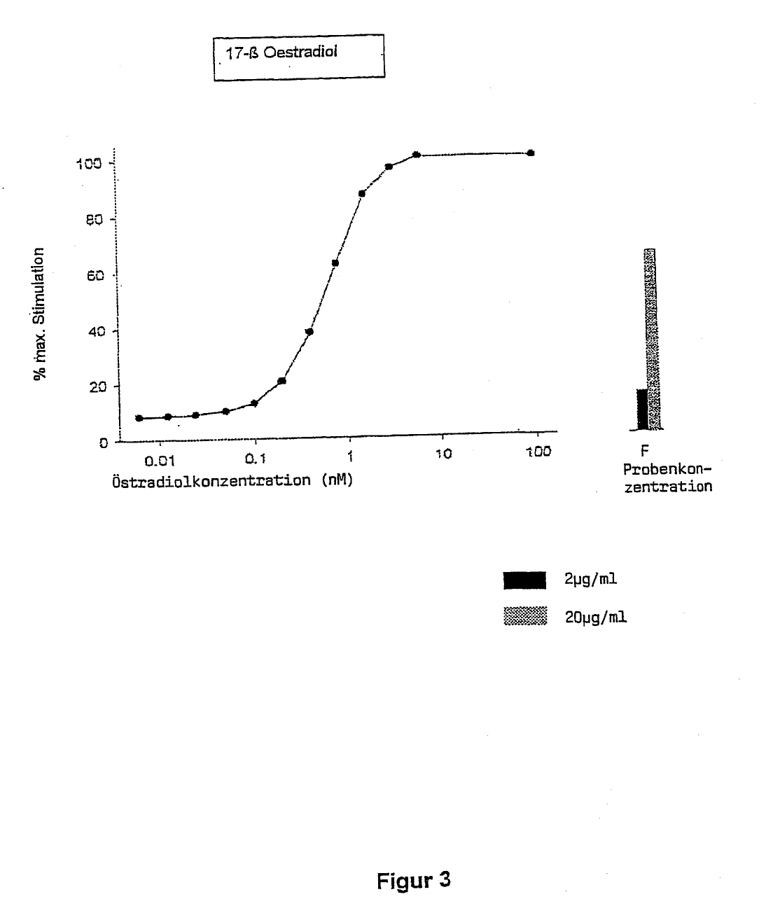

FIG. 3 illustrates the oestrogenic action of extracts according to the invention (60% [w/w] ethanol extracts after distribution with ethylacetate according to example 3) from Sophora flavescens in a yeast assay, compared with 17β oestradiol.

Similar extracts can generally be obtained by extraction with a solvent of medium polarity, chosen from the group consisting of aqueous alcohols, aqueous ketones and esters, including aqueous or water-saturated esters, draining off the solvent and subsequent fluid-fluid distribution between an organic solvent and water.

The alcohols or ketones can be used as 10-96% [v/v] or [w/w/], or 10-99% [v/v] or [w/w], particularly 50-92% [v/v] or [w/w] aqueous mixtures, wherein 70% [v/v] (62% [w/w]) ethanol, 60% [w/w] ethanol or water-saturated ethylacetate is particularly preferred.

For the liquid-liquid-distribution organic solvents can be used selected from the group comprising ethylacetate, tert-butylmethylether, n-butanol and butanon with ethylacetate being preferred.

The extracts obtainable under the invention are characterised in that they have a total alkaloid content below 0.2%, and preferably below 0.1%. In particular, the extract is free of alkaloid. In this context, the term “alkaloid-free” means that it is undetectable using standard analytical procedures such as HPLC.

The extracts of Sophora flavescens and Sophora subprostrata are used according to the invention for the prophylaxis and treatment of pathological conditions caused by oestrogen deficiency or by a dysregulation of the sex-hormone-related metabolism, in particular oestrogen metabolism.

The pathological conditions can be selected from those belonging to the group consisting of climacteric complaints, sex hormone-dependent cancers, benign prostate hyperplasia, osteoporosis, Alzheimer's disease and cardiovascular diseases: the cancers included here are breast cancer, prostate cancer and cancer of the womb.

The extracts obtained according to the invention can be processed together with conventional pharmaceutically-acceptable additives to pharmaceutical preparations such as capsules, film tablets or coated tablets, wherein as conventional pharmaceutically-acceptable additives fillings, bonding agents, spreaders and coatings for film and coated tablets, as well as oil or fat as excipients for capsules, can be used.

The extracts according to the invention are applied to humans at a dosage from 1-1000 mg daily, preferably 100-1000 mg daily.

The following examples describe the invention and should not be considered to limit the invention. All percentage details refer to the weight, unless specified otherwise.

### EXAMPLE 1

Production of an Extract from Sophora flavescens

a) Extraction

2 kg of ground Sophora flavescens roots were mixed with 14 kg 70% [v/v] (62% [w/w]) ethanol, extracted by spinning (Ultraturrax) for 5 min and then stirred intensively for 1 hr at 50° C. Then it was filtered with suction over a Supra Seitz 1500 filter and the drug residue was then extracted for a second time in the same way. Both the extract solutions were combined and the dry extract content was determined using an aliquot. It turned out that there was a dry residue of 475.6 g, corresponding to a yield of 23.8%.

b) Distribution

The ethanol was removed from the extract solution on the rotary evaporator at 50° C. The watery residue (5 l) was stirred three times with 2 l ethylacetate each time, the ethylacetate phases were combined and concentrated in the rotary evaporator till dry.

Ethylacetate extract=67.84 g (3.4% by reference to the drug; 14.3% by reference to total extract).

According to the HPLC analysis, the ethylacetate extract contains flavones, isoflavones, chalcones, pterocarpanes (e.g. 8-prenylnaringenine, daidzein, kushenol X, norkurarinon, macchian, genistone, 8-prenylcamphorol). There was no evidence of alkaloids.

This combination of substances is preferably suited to the prophylaxis of and treatment of pathological conditions caused by oestrogen deficiency or by dysregulations to sex hormone-related metabolism, in particular oestrogen metabolism (compare example 5).

### EXAMPLE 2

Production of an Extract from Sophora subprostrata.

a) Extraction

1 kg of ground S. subprostrata roots was mixed with 7 kg 70% [v/v] (62% [w/w]) ethanol, extracted by spinning (Ultraturrax) for 5 minutes and then stirred intensively at 50° C. for 1 hour. Then it was filtered with suction over a Supra Seitz 1500 filter and the drug residue was then extracted for a second time in the same way. Both the extract solutions were combined and the dry extract content was determined using an aliquot. It turned out that there was a dry residue of 111.8 g, corresponding to a yield of 11.2%.

b) Distribution

The ethanol was removed from the extract solution on the rotary evaporator at 50° C. The watery residue (2.5 l) was stirred thrice with 1 l ethylacetate each time, the ethylacetate phases were combined and concentrated in the rotary evaporator till dry.

Ethylacetate extract=10.4 g (1.0% by reference to the drug; 9.3% by reference to total extract)

### EXAMPLE 3

36 kg of ground Sophora flavescens drug was mixed with 7.7 times its weight made up of 60% (w/w) ethanol. Given vigorous stirring and with Dispax (Ultraturrax) in circulation, the extract solution was extracted for 1 hr at 50° C. The extract solution was filtered clear, the drug residue was once again extracted with 7 times its weight made up of 60% (w/w) ethanol under the same conditions, and finally filtered.

The extract solution was then concentrated in the centrifugal evaporator to an ethanol content of 3.5% and dry extract content of 10.52%. This concentrated solution was then distributed against water-saturated ethylacetate three times at a 1:0.4 ratio by volume. The ethylacetate phase was concentrated at the rotary evaporator and dried out in the drying cupboard at 600° C.

This resulted in:

Ethylacetate extract: 1.188 kg after grinding (3.3% by reference to the drug).

HPLC Content of Pharmacologically Relevant Substances:

| | |
|---|---|
| Macchian glucoside | 2.8% |
| 8-prenylnaringenine | 0.4% |
| Macchin | 0.6% |
| Kushenol X | 1.3% |
| 8-prenylcamphorol | 0.7% |
| Leachianon G | 6.6% |
| Kushenol E | 0.3% |
| 2,4,4′,6′-tetrahydroxy-3′- | 0.7% |
| lavandulyl-2′-methoxychalcone | |

There was also evidence of daidzein and genistone. There was no evidence of any alkaloids.

Estrogenic action could be shown for each of the substances mentioned in a yeast assay according to example 5. The oestrogenic action for the extract is produced by the Reportergen assay with the use of yeast cells according to example 5; compare FIG. 3.

HPLC Method

| Columns | LiChrosphere 100 5 μm, 250 × 4 mm | |
|---|---|---|
| Eluens | A: 1000 ml bidest water/3 ml phosphoric acid | |
| | (85%)/2 ml triethylamine | |
| | B: 100 ml acetonitril/3 ml phosphoric acid | |
| | (85%)/2 ml triethylamine/60 ml bidest water | |
| Gradient | % A | % B |
| | 0 min 70 | 30 |
| | 30 min 30 | 70 |
| | 35 min 0 | 100 |
| Flow | 1.2 ml/min | |
| Detection | 220 nm | |

### EXAMPLE 4

150 g of ground Sophora flavescens drug was extracted twice with water-saturated ethylacetate at the ratio of 1:7 (m/m). Therefore, the drug was broken down beforehand with an Ultra-Turrax for five minutes (extraction by movement). The drug was then extracted under reflux for 1 hour at 60° C. The combined extract solutions were then filtered off and the ethylacetate extract solution thus obtained was shaken back twice with ethylacetate-saturated water at the ratio 1:1 {v/v]. The combined ethylacatate phases were concentrated till dry.

Yield: 3.99% by reference to the drug.

### EXAMPLE 5

Testing the Ethylacetate Extract Produced for Oestrogenic Action with Transfected Yeast Cells, which Express the Human α Oestrogen Receptor.

The testing of extracts for oestrogenic properties was performed with a Reportergen assay, using yeast cells (saccharomyces). These cells are stable with the human α oestrogen receptor and an expression plasmid which contains an oestrogen response element and the gene for the enzyme β galactosidase. All samples were dissolved in DMSO at a concentration of 20 mg/ml, and were given undiluted or after diluting with DMSO at the ratio of 1/10, 1/100 or at a volume of 1 μl to 100 μl culture medium in 96-well flat-bottom micro-titre dishes. Next, 100 μl yeast suspension and the chromogenous substrate chlorophenolred-β-D-galactopyranoside were added. Control wells were provided on every dish, which were filled with either the culture medium or the solvent alone, or which contained the standard concentrations of 17β oestradiol. The yeast cells were incubated for 72 hours at 320° C., after which absorption of the medium was measured at 540 nM in a micro-titre dish photometer. The samples were sometimes checked twice.

Results:

Sample Concentrations:

| Dilution | Concentration |
|---|---|
| undiluted | 100 μg/ml |
| 1:10 | 10 μg/ml |
| 1:100 | 1 μg/ml |

Designation of Samples:

| Sample | | Number of | |
|---|---|---|---|
| number | Sample | tests | Sophora species |
| A | 70% [v/v] (62% [w/w]) | 1 | Sophora flavescens |
| | ethanol extract | | |
| | according to ex. 1a) | | |
| B | 70% [v/v] (62% [w/w]{ | 1 | Sophora subprostrata |
| | ethanol extract | | |
| | according to ex. 2a) | | |
| C | 70% [v/v] (62% [w/w] | 2 | Sophora flavescens |
| | ethanol extract | | |
| | according to ex. 1a) | | |
| D | Ethylacetate extract | 1 | Sophora flavescens |
| | according to ex. 1b) | | |
| E | Ethylacetate extract | 1 | Sophora subprostrata |
| | according to ex. 2b) | | |
| F | Ethylacetate extract | 1 | Sophora flavescens |
| | according to ex. 3 | | |

The results of the assays are depicted in FIGS. 1, 2 and 3. In so doing, the oestrogenic action of the extracts was compared against the action of 17β oestradiol. For this purpose, extracts that can be designated as “active” are those whose action when compared with 17β oestradiol matches the action of 17β oestradiol that has become established within the distinctly rising segment of the 17β oestradiol activity curve (and hence from a concentration of around 0.1-0.2 nM of 17β oestradiol).

In particular, FIG. 1 illustrates the oestrogenic action of 70% [v/v] (62% [w/w]) ethanol extracts (according to examples 1a) and 2a)) from Sophora flavescens and Sophora subprostrata in a yeast assay, compared with 17β oestradiol.

FIG. 2 illustrates the oestrogenic action of the extracts under this invention (70% [v/v] or 62% [w/w] ethanol extract after distribution with ethylacetate; according to examples 1b) and 2b)) from Sophora flavescens and Sophora subprostrata in a yeast assay, compared with 17β oestradiol.

FIG. 3 illustrates the oestrogenic action of extracts according to the invention (60% [w/w] ethanol extracts after distribution with ethylacetate according to example 3) from Sophora flavescens in a yeast assay, compared with 17β oestradiol.

1. Use of an extract from Sophora flavescens or Sophora subprostrata for the preparation of a medicament for the prophylaxis and treatment of pathological conditions caused by a deficiency of oestrogens or by a dysregulation of sex hormone metabolism, particularly oestrogen metabolism.

2. Use according to claim 1, wherein the pathological condition is selected from the group consisting of climacteric complaints, benign prostate hyperplasia, osteoporosis, Alzheimer's disease and cardiovascular diseases.

3. Method for the prophylaxis and treatment of pathological conditions caused by a deficiency of oestrogens or by a dysregulation of sex hormone metabolism, particularly oestrogen metabolism, wherein the method comprises the administration of an active amount of an extract from Sophora flavescens or Sophora subprostrata.

4. Method for the prophylaxis and treatment of pathological conditions caused by a deficiency of oestrogens or by a dysregulation of sex hormone metabolism, particularly oestrogen metabolism, wherein the method comprises the administration of an active amount of a pharmaceutical preparation containing an extract from Sophora flavescens or Sophora subprostrata.

5. Method according to claim 3 or 4, wherein the pathological condition is selected from the group consisting of climacteric complaints, benign prostate hyperplasia, osteoporosis, Alzheimer's disease and cardiovascular diseases.
